(19) United States
(12) Patent Application Publication
(10) Pub. No.: US 2025/0040817 A1
(43) Pub. Date: Feb. 6, 2025

(54) BIODEGRADABLE BLOOD PRESSURE CUFF

(71) Applicant: Bannack Medical LLC, Gilbert, AZ (US)

(72) Inventors: Arlen Olsen, Gilbert, AZ (US); Richard Mayer, Chandler, AZ (US)

(21) Appl. No.: 18/393,254

(22) Filed: Dec. 21, 2023

Related U.S. Application Data
(60) Provisional application No. 63/531,639, filed on Aug. 9, 2023.
(60) Provisional application No. 63/536,015, filed on Aug. 31, 2023.
(60) Provisional application No. 63/528,602, filed on Jul. 24, 2023.

(51) Int. Cl. A61B 5/022; A61L 31/04; A61L 31/16

(52) U.S. Cl. CPC A61B 5/02233; A61L 31/041; A61L 31/16; A61L 2300/404

(57) ABSTRACT

A blood pressure cuff is disclosed having an inflatable bladder of: a non-biodegradable polymer selected from a group consisting of polyamide, polyester, polyethylene (PE), polypropylene (PP), polyvinylchloride (PVC), polyurethane and combinations thereof; and an additive comprising a blend or copolymer of (1) a first polymer selected from a poly-lactic acid (PLA), a polyhydroxyalkanoates (PHA) or a combination thereof and (2) a second polymer which is a poly-terephthalate. The blood pressure cuff may be single-use of reusable.

This application claims priority to provisional patent application No. 65/531,639 filed on Aug. 9, 2023; provisional patent application No. 63/536,015 filed on Aug. 31, 2023; and provisional patent application No. 63/528,602 filed Jul. 24, 2023 all of which are hereby incorporated by reference.

### FIELD OF THE INVENTION

The invention is a biodegradable blood pressure cuff, most often, for use in health care, to maintain a clean environment and patient safety due to body fluids sustained from injury or infectious diseases while meeting hospital sustainability goals. In particular, it relates to reusable and disposable blood pressure cuffs.

### BACKGROUND

Disposal of plastic waste is a serious environmental problem. Increased use of plastics and other synthetic materials has resulted in a growing environmental impact. To combat this, bioplastics such as biodegradable polymers are being developed to be used as an alternative for non-biodegradable polymer materials. The best option for managing non-biodegradable plastic waste is to replace non-biodegradable materials with biodegradable polymers as they are environmentally friendly. In some cases, non-biodegradable plastics may be recycled. Many of the products that hospitals and healthcare providers use are disposable plastic products that are non-biodegradable. This is a problem and a growing concern for hospitals and healthcare facilities meeting sustainability goals.

Recently, healthcare providers are moving away from traditional sphygmomanometers or blood pressure cuffs that are used over and over again on patients due to their harboring infectious diseases and causing cross-contamination to patients. Friedman, Bruce. 2020. Improving Quality of Care: Justifying the Cost for a Single-Patient-Use Blood Pressure Cuff. See https://clinicalview.gehealthcare.com/white-paper/improving-quality-care-justifying-cost-single-patient-use-blood-pressure-cuff Instead, they are moving toward a hospital disposable, such as a single patient disposable blood pressure cuff and/or a reusable blood pressure cuff. With increased demand for single patient disposable blood pressure cuff, this creates a problem for being able to dispose of the single patient disposable blood pressure cuff after use. Many solutions are being considered other than placing them in landfills, such as recycling by melting the support surface down. This creates the challenge of having the healthcare workers obtaining adequate recycling solutions. Many hospitals and healthcare workers are also under pressure to come up with sustainability solutions for hospital waste.

### SUMMARY

The disclosed single patient, single use, and/or reusable biodegradable disposable blood pressure cuff modifies the materials used in the components of the disposable blood pressure cuff with biodegradable plastics. The biodegradable blood pressure cuff helps reduce the risk of cross-contamination and environmental impact. The disclosed has been lab tested using ASTM D5511, showing faster results for biodegradable plastic than conventional materials used. For example, an additive will be added to the material used to manufacture conventional blood pressure cuffs including; for example, the bladder, the connectors and the tubing can be a polymer made with 99% polymer and 1% of the additive.

In a first aspect is a blood pressure cuff comprising: an inflatable bladder comprised of: a non-biodegradable polymer selected from a group consisting of polyamide, polyester, polyethylene (PE), polypropylene (PP), polyvinylchloride (PVC), nylon, polyurethane and combinations thereof; and an additive comprising a blend or copolymer of (1) a first polymer selected from a poly-lactic acid (PLA), a polyhydroxyalkanoates (PHA) or a combination thereof and (2) a second polymer which is a poly-terephthalate.

In a second aspect is a blood pressure cuff comprising: an inflatable bladder; and at least one connector, wherein the inflatable bladder and the at least one connector are made from a material comprised of: a non-biodegradable polymer selected from a group consisting of polyamide, polyester, polyethylene (PE), polypropylene (PP), polyvinylchloride (PVC), nylon, polyurethane and combinations thereof; and a biodegradable additive.

In a third aspect is a single patient blood pressure cuff comprising: an inflatable bladder selected from polyamide, polyester, polyethylene (PE), polypropylene (PP), polyvinylchloride (PVC) and combinations thereof; at least one connector selected from a group consisting of nylon, polyester, polyethylene (PE), polypropylene (PP), polyvinylchloride (PVC), nylon, polyurethane and combinations thereof; tubing selected from a group consisting of polyvinyl chloride, polyethylene, polypropylene, polyurethane, polyamide, polytetrafluoroethylene, polyvinylidene fluoride, ethylene vinyl acetate, polycarbonate or combinations thereof; and an additive added to each of the materials in a-c comprising a blend or copolymer of (1) a first polymer selected from a poly-lactic acid (PLA), a polyhydroxyalkanoates (PHA) or a combination thereof and (2) a second polymer which is a poly-terephthalate.

### BRIEF DESCRIPTION OF THE DRAWINGS

FIG. 1 shows a first side of a biodegradable blood pressure cuff; and

FIG. 2 shows a first side of a biodegradable blood pressure cuff.

### DETAILED DESCRIPTION

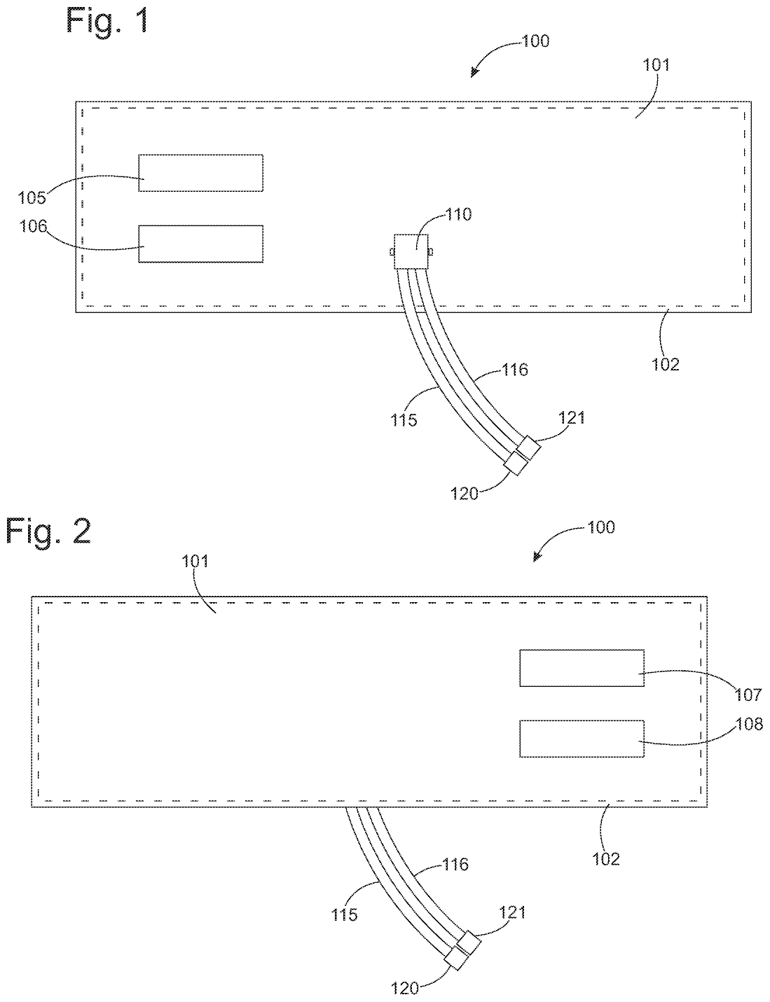

FIGS. 1 and 2 show a disposable and/or single patient and/or reusable blood pressure cuff. FIG. 1 shows a first side and FIG. 2 shows a second side. The blood pressure cuff 100 is made from one of a nonporous polyamide (nylon), polyester, polyethylene (PE), polypropylene (PP), polyvinylchloride (PVC), polyurethane and combinations thereof made with an additive as hereinafter described. The blood pressure cuff 100 includes an inflatable bladder 101. The inflatable bladder can come in various sizes and with various connector options to be adapted to most systems. The inflatable bladder 101 has at least two layers that are heat sealed, bonded, melted, glued or otherwise adhered together 102 to form a bladder. Alternatively, the inflatable bladder 101 can be a tube with ends bonded together to form the bladder or may be manufactured as a single bladder. The bladder includes a hook and loop fastener with a first pair strips shown at 105 and 106 and a second pair shown at 107 and 108. Alternatively, straps may be used with fasteners thereon desirably made of nylon.

In the case of reusable blood pressure cuffs, the cuff material 200 is desirably made of a polyamide, but can also be made of a polyester, polyethylene (PE), polypropylene (PP), polyvinylchloride (PVC), polyurethane and combinations. The cuff 200 will cover a gas bag typically made of polyethylene, polypropylene, polyvinylchloride (PVC) or polyurethane but could also be made of polyamide, polyester, and combinations

A cuff connector 110 is shown which attaches to a nipple, luer lock, push-type, Neo-quick® or other mating connector on the inflatable bladder 101. The air pump connectors 120, 121 are attached to end of tubing 115 and 116. The cuff connector 110, air pump connectors are an injection molded part made from a polyamide, polyester, polyethylene (PE), polypropylene (PP), polyvinylchloride (PVC), polyurethane and combinations thereof made with an additive as hereinafter described. The tubing 115, 116 may be made from polyvinyl chloride, polyethylene, polypropylene, polyurethane, polyamide, polytetrafluoroethylene, polyvinylidene fluoride, ethylene vinyl acetate, polycarbonate or combinations thereof made with an additive as hereinafter described.

The cuff connector 110 may be a luer lock, push-type, Neo-quick®, etc. The various connectors for a one-tube machine are screw connector, tri-purpose connector, locking connector, and bayonet connector; and a two-tube machine are a screw connector, locking connector, tri-purpose and empty tube connector, and inflation system.

In use, the disposable and/or single patient and/or reusable blood pressure cuff 100 is attached to valve mechanism (not shown) to control inflation and deflation or the inflatable bladder 101. An air pump (not shown) can be manual or automatic can be used for the inflatable bladder 101 to fill it with air. A pressure gauge (analog or digital) (not shown) is used to read the blood pressure of the patient. Blood pressure cuffs do not directly measure systolic or diastolic blood pressures. Instead the cuff only measures directly the mean arterial pressure through an oscillometric technique. Initially the cuff occludes the brachial artery to prevent flow. As the cuff pressure drops, turbulent flow is generated through the blood vessel creating oscillations against the arterial wall. Through a stethoscope the health care provider can notice sound of the blood flow. The first pulse is the systolic pressure. As soon as no more heart pulse beat can be heard that is the diastolic pressure. As the pressure keeps dropping, the oscillations reach a point of maximal amplitude. The cuff then fully deflates opening up the artery to provide more laminar flow and reducing oscillations.

The various components will have different durometer hardness between the fabric of the inflatable bladder 101, the connectors 110, 115, 116 and the tubing 115, 116. Each of these components materials will have the additive added to their material. However, it's important to note that the actual hardness can vary depending on the specific manufacturing processes and additives used in the production of each material. Different grades and formulations can result in variations in hardness, even within the same category of material. In the embodiments of the invention, it is important that connectors and tubing are much more rigid than the cuff so that the connectors can firmly attach the single patient disposable blood pressure cuff in place with the tubing. For example, hardware, i.e. connectors will range between Shore D 45 to Shore D80 with a desired range of Shore D 50 to Shore D80 and a most desired Shore D 70 wherein in this range it will strike a balance between flexibility and stiffness. While offering a good durability and still maintaining some flexibility, making them suitable for various hardware applications.

The inflatable bladder is softer and more pliable and would be on the Shore A scale which is used to measure the hardness of soft pliable material. Shore A 30 to Shore A 70, the most desirable is Shore A 60 to Shore A 70 with a Shore A of 65. An inflatable bladder with durometer values in this range are very soft and pliable. Nonwoven polypropylene parts with such hardness values are often used in applications where the material needs to be gentle against the skin. Parts with durometer values in this range are considered relatively soft and flexible. They offer good flexibility, drape, and conformability, making them suitable for wrapping around the arm or leg of a patient. The tubing has a range of Shore A 30 to Shore A 70, the most desirable is Shore A 60 to Shore A 70 with a Shore A of 65.

One embodiment of the invention would include adding an antimicrobial to the manufacturing process of the blood pressure cuff. The most common additives used to manufacture antimicrobial plastics include various isothiazolinone treatments, zinc pyrithione, thiabendazole, and silver antimicrobial products. Each active ingredient has its strengths and weaknesses.

Although polypropylene is the desired material in combination with a biodegradable additive, an aspect disclosed in embodiments of the invention are hospital disposables made of one or more polymers (e.g. polyamide, polyester, polyethylene (PE), polypropylene (PP), polyvinylchloride (PVC), polyurethane and combinations thereof) and an additive comprising a blend or copolymer of (1) a first polymer selected from a poly-lactic acid (PLA) and/or a polyhydroxyalkanoates (PHA) and (2) a second polymer which is a poly-terephthalate. In the additive, the first polymer and second polymer are covalently bound to one another to form a copolymer. In another embodiment, the first polymer and second polymer are blended together to form an admixture but are not covalently bound to one another.

In one embodiment, the base polymer of polyamide, polyester, polyethylene (PE), polypropylene (PP), polyvinylchloride (PVC), polyurethane and combinations thereof is present in the hospital disposable at a concentration of 90-99.9 wt. %, at a concentration of 95-99.5 wt. %, at a concentration of 98-99.5 wt. % or at a concentration of 99 wt. %, with the mass balance being the additive. In such embodiments, the additive is present at a concentration of 0.1-10 wt. %, 0.5-5 wt. %, 0.5-2 wt. % and 1% wt. %, respectively.

The first polymer of the additive is a PLA, a PHA or a combination thereof. The term PLA includes poly-D-lactic acid (PDLA), poly-L-lactic acid (PLLA) and combinations thereof. Specific examples of PHAs include poly-3-hydroxybutyrate (PHB), poly-3-hydroxybutyrate-co-4-hydroxybutyrate (P(3-HB-co-4-HB)), poly-3-hydroxybutyrate-co-valerate (PHBV), and polyhydroxybutyrate-co-hexanoate (PHBH). In one embodiment, the first polymer is PLA sold under the brand name INGEO® and has a number-average molecular weight of 127 kg per mole and a polydispersity index of 1.6. In other embodiments, the number-average molecular weight is between 100-150 kg per mole.

| PLA | PHA |
|---|---|
| 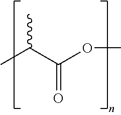 | 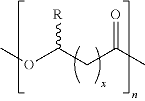 |
| | X is an integer from 1-5 |
| | R is an alkane or branched alkane including |
| | methyl, ethyl, propyl, isopropyl, butyl and |
| | isobutyl |

The second polymer of the additive is a poly-terephthalate. Examples include copolymers such as polybutylene adipate terephthalate (PBAT), polybutylene terephthalate, polycyclohexylenedimethylene terephthalate, polyethylene terephthalate, polytrimethylene terephthalate, poly(butylene succinate terephthalate) (PBST) and poly(butylene sebacate terephthalate). In one embodiment, the second polymer is a PBAT polymer sold under the brand name ECOFLEX® and has a number-average molecular weight of about 52 kg per mole and a polydispersity index of 2. In other embodiments, the number-average molecular weight is between 40 and 60 kg per mole.

In one embodiment, the additive comprises a blend of 30-70 wt. % of the first polymer and 30-70 wt. % of the second polymer. In another embodiment, the additive comprises a blend of 40-60 wt. % of the first polymer and 40-60 wt. % of the second polymer. In another embodiment, the additive comprises a blend of 45-55 wt. % of the first polymer and 45-55 wt. % of the second polymer. In another embodiment, the additive comprises a blend of 49-51 wt. % of the first polymer and 49-51 wt. % of the second polymer. In yet another embodiment, the additive consists of a blend of 49-51 wt. % of the first polymer and 49-51 wt. % of the second polymer.

In one embodiment, the second polymer is a blend/admixture of a polyester (1) and a poly-terephthalate (2), the structure of which are shown below. In another embodiment, the second polymer is a copolymer prepared by transesterifying polyester (1) and poly-terephthalate (2). The values of a, b and c are integers independently selected from 1-8. In one embodiment, a and c are integers from 1-6 and b is an integer from 1-8. In one embodiment, a and c are both 4. In one embodiment, b is an integer from 1-4. In another embodiment, b is an integer from 5-8. In one embodiment, the values of a, b and c are all four. The transesterification is performed using conventional methods including acid catalyzed transesterification. Organometallic catalysts are known for facilitating such a reaction including tetrabutoxytitanium and other zinc, tin and germanium-based catalysts. The transesterification may be performed at high temperature (e.g. greater than 190° C.) and under vacuum to facilitate removal of volatile byproducts, including water. During transesterification, random block copolymerization of the monomeric units may occur.

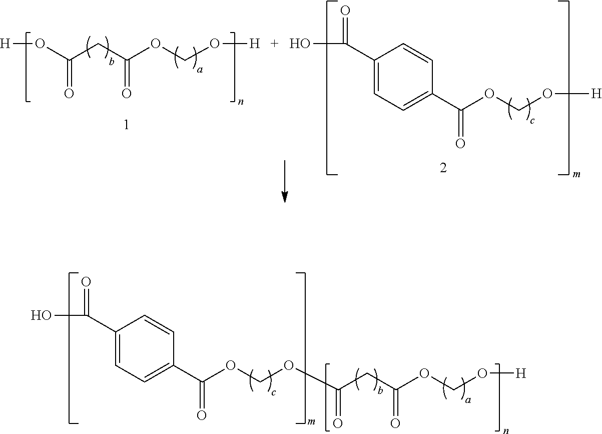

In one embodiment, a=b=c=4 and the poly-terephthalate (2) is polybutylene adipate terephthalate (PBAT).

In one embodiment, the values of m and n are within 30% of one another (e.g. a m:n molar ratio of 1.3:1 to a ratio of 1:1.3. In another embodiment, the values of m and n are within 20% of one another (e.g. a m:n ratio of 1.2:1 to 1:1.2). In another embodiment, the values of m and n are within 10% of one another (e.g. a m:n ratio of 1.1:1 to 1:1.1). In another embodiment, the values of m and n are within 5% of one another (e.g. a m:n ratio of 1.05:1 to 1:1.05). In another embodiment, the values of m and n are within 1% of one another (e.g. a m:n ratio of 1.01:1 to 1:1.01). In one embodiment, the terephthalate monomer is present in the additive at a concentration of less than 55 mol %.

The polyester (1) may be prepared from an alkane diol (3) and a diacid (R₁═H) (4). In one embodiment, R₁ in the diacid (4) is methyl, ethyl or propyl such that a corresponding diester is used. In one embodiment, a is four such that compound (3) is 1,4-butane diol. In some embodiments, R₁ is hydrogen and b is four (adipic acid), two (succinic acid) or eight (sebacic acid).

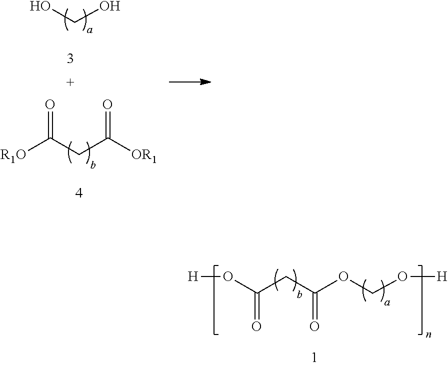

In one embodiment, R₁ is hydrogen and b is four (i.e. adipic acid) and a is four (i.e. 1,4-butane diol) as shown below.

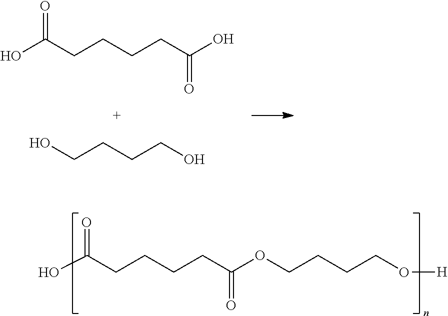

The poly-terephthalate (2) is prepared from a terephthalate ester (5) (or its corresponding acid) and alkane diol (6). In terephthalate ester (5) R₁ may be H or an alkane such as methyl, ethyl, propyl, isopropyl, etc. The value of c is an integer from 1-6. In one embodiment, c is four such that compound (6) is 1,4-butane diol.

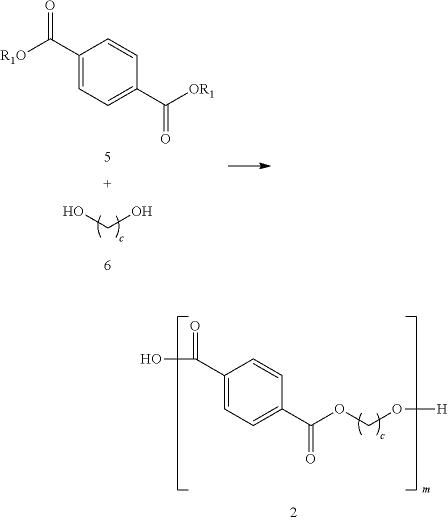

In one embodiment, R₁ is methyl and c is four, as shown below.

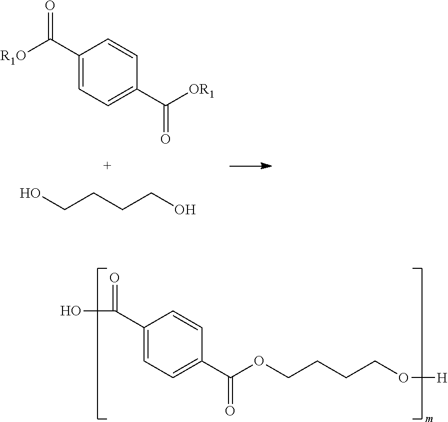

The additive has a crosslinking density that renders it biodegradable. In one embodiment, the crosslinking density is less than 30%, less than 20% or less than 10%. The crosslinking density can be determined by using ASTM-D2765. This method determines gel content and swell ratio for a known mass of polymer extracted with a suitable solvent. The extracted material is separated from the solvent and weighed after drying. The higher the mass of extracted material, the lower the crosslinking density.

The additive has a glass transition temperature (T_{g}) within or below the optimal temperature of mesophilic bacteria (e.g. a T_{g} within a temperature range of 20° C. to 45° C.).

In particular embodiments, the hospital disposable such as nylon fabric and an additive comprising a blend of poly-L-lactic acid (PLLA) and a poly-terephthalate. Another aspect is a hospital disposable made of 90-99.9 wt. % polypropylene and 0.1-10 wt. % of a blend of poly-L-lactic acid and a poly-terephthalate, or 95-99.5 wt. % polypropylene and 0.1-5 wt. % of a blend of poly-L-lactic acid and a poly-terephthalate, or 98-99.5 wt. % polypropylene and 0.1-2 wt. % of a blend of poly-L-lactic acid and a poly-terephthalate, or 99 wt. % polypropylene and 1 wt. % of a blend of poly-L-lactic acid and a poly-terephthalate.

A further aspect of the embodiments is an additive for use with nylon comprising a blend of 30-70 wt. % poly-L-lactic acid and 30-70 wt. % poly-terephthalate, or an additive for use with polypropylene comprising a blend of 40-60 wt. % poly-L-lactic acid and 40-60 wt. % poly-terephthalate, or an additive for use with polypropylene comprising a blend of 45-55 wt. % poly-L-lactic acid and 45-55 wt. % poly-terephthalate, or an additive for use with polypropylene comprising a blend of 49-51 wt. % poly-L-lactic acid and 49-51 wt. % poly-terephthalate. Another aspect is a method of making a hospital disposable comprising the steps of: blending polypropylene with an additive of poly-L-lactic acid and a poly-terephthalate; and at least one of extruding, molding and forming a hospital disposable from the blend.

### Example 1

99 wt. % polypropylene was mixed/blended with 1 wt. % additive comprised of 51 wt. % poly-L-lactic acid (PLLA) polymer and 49 wt. % poly(butylene adipate-co-terephthalate) (PBAT), or a copolymer of the two for 5 minutes. The mixture/blend was then formed into biodegradable polypropylene fabric for use as a blood pressure cuff.

### Example 2

In another example, a cuff connector and air pump connectors, and tubing are produced with 99 wt. % polypropylene was mixed/blended with 1 wt. % additive comprised of 51 wt. % poly-L-lactic acid (PLLA) polymer and 49 wt. % poly(butylene adipate-co-terephthalate) (PBAT), or a copolymer of the two for 5 minutes. The mixture was then injection molded to product a cuff connector and air pump connectors. The a cuff connector and air pump connectors have a durometer hardness different than the blood pressure cuff. The injection molding proceeded as follows:

Material Preparation: Polypropylene pellets or granules are selected as the raw material. Additive pellets of 51 wt. % poly-L-lactic acid (PLLA) polymer and 49 wt. % poly(butylene adipate-co-terephthalate) (PBAT) were added. Each of these pellets are fed into the injection molding machine's hopper.

Heating and Melting: The polypropylene and additive pellets are heated and melted within the injection molding machine's barrel. The temperature is carefully controlled to ensure proper melting without causing degradation of the plastic material.

Injection: Once the polypropylene and additive is molten, it is injected under high pressure into the mold cavity using a reciprocating screw or a plunger. The mold is closed and clamped to prevent any leakage during the injection process.

Cooling and Solidification: The molten polypropylene and additive quickly starts to cool down as it comes into contact with the mold surfaces. Cooling channels within the mold help expedite the cooling process. As the plastic cools, it solidifies and takes on the shape of the buckle.

Mold Opening and Ejection: Once the polypropylene and additive has solidified, the mold is opened, revealing the molded cuff connector and air pump connectors. Ejection pins or mechanisms help push the cuff connector and air pump connectors out of the mold.

Trimming and Quality Control: Any excess material or flash around the edges of the buckle is trimmed off. The buckle is then inspected for defects, proper dimensions, and overall quality. This step ensures that the final product meets the required specifications.

The cuff connectors and air pump connectors produced by Example 2 was then assembled onto the inflatable bladder of Example 1 to provide a biodegradable blood pressure cuff.

The tubing may be manufactured using extrusion. Extrusion is a widely used method in the plastics industry to create continuous lengths of plastic products with a consistent cross-sectional shape. Here's how extruded plastic tubing is made:

Material Preparation: 99 wt. % polypropylene was mixed/blended with 1 wt. % additive comprised of 51 wt. % poly-L-lactic acid (PLLA) polymer and 49 wt. % poly(butylene adipate-co-terephthalate) (PBAT), or a copolymer of the two for 5 minutes. The is typically loaded into a hopper that feeds into the extrusion machine.

Melting and Mixing: The admixed pellets are fed from the hopper into the extruder, which consists of a rotating screw within a heated barrel. As the pellets move through the extruder, the heat generated by friction and the heated barrel melts the plastic into a viscous, molten state.

If desired, additives such as colorants, stabilizers, anti-bacterials or UV protectants can be introduced into the molten plastic to achieve specific characteristics.

Extrusion Process: The molten plastic is forced through a specially designed die at the end of the extruder. The die determines the shape and dimensions of the tubing's cross-section. As the molten plastic emerges from the die, it forms a continuous shape, similar to the desired final tubing profile.

Cooling and Sizing: The extruded plastic tubing is then passed through a cooling system, which can involve air or water cooling. This helps to solidify and stabilize the shape of the tubing.

Pulling and Cutting: A pulling mechanism, often using rollers or a belt system, is used to draw the extruded tubing at a controlled speed. Downstream, the tubing is cut into specific lengths using cutting devices, such as rotary cutters or guillotine-style cutters.

Examples of various embodiments are provided herein below.

### Example 1: Process of Making a Reusable Blood Pressure Cuff of Nylon Fabric

99 wt. % nylon was mixed/blended with 1 wt. % additive comprised of 51 wt. % poly-L-lactic acid (PLLA) polymer and 49 wt. % poly(butylene adipate-co-terephthalate) (PBAT), or a copolymer of the two for 5 minutes. The mixture/blend was then formed into nylon fabric.

Nylon fabric is a synthetic fabric made from polyamide fibers, and its creation involves several steps which are described herein below:
1. Polymerization: The first step is to create the nylon polymer, which is achieved through a process called polymerization. The main raw materials used in the polymerization of nylon are petroleum-derived chemicals, such as adipic acid and hexamethylenediamine. These chemicals react together under controlled conditions to form a liquid polymer called nylon salt.
2. Condensation: The nylon salt is then heated to high temperatures, causing it to undergo a condensation reaction. During this reaction, the nylon salt transforms into a solid nylon polymer. The nylon polymer is usually in the form of chips or pellets at this stage.
3. Melting and Extrusion: The solid nylon polymer is then melted and forced through spinnerets, which are small metal plates with tiny holes. The shape and size of the holes in the spinnerets determine the thickness and texture of the resulting nylon fibers. As the molten nylon polymer is extruded through the spinnerets, it solidifies and takes the form of long, continuous filaments.
4. Drawing: After extrusion, the nylon filaments are stretched or drawn to align the molecules and improve the strength and durability of the fabric. Drawing also affects the final texture of the fabric, making it smoother and more uniform.
5. Cooling and Solidification: The drawn nylon filaments are rapidly cooled to solidify them in their stretched state. This process helps to lock in the molecular alignment achieved during drawing.
6. Crimping (Optional): In some cases, the nylon filaments are crimped to introduce texture and bulkiness to the fabric, which is desirable for certain applications.
7. Spinning into Yarn: The solidified nylon filaments are then wound onto bobbins to form yarn. The yarn can be used in its pure form or blended with other fibers to create various types of nylon fabrics with different characteristics.
8. Weaving or Knitting: The nylon yarn is then woven or knitted into fabric on industrial looms or knitting machines. The weaving or knitting process creates the final fabric with the desired pattern and texture.
9. Finishing: After weaving or knitting, the nylon fabric undergoes various finishing processes to enhance its properties, such as dyeing, printing, and coating to make it water-resistant or flame-retardant. A biodegradable polyurethane and/or biodegradable water-resistant coating may be sprayed thereon.

The resulting nylon fabric is lightweight, strong, abrasion-resistant, and has excellent elasticity, making it suitable for use with a lateral transfer support surface.

### Example 2: Process of Making Polypropylene Cuff Connector and a Nipple, Luer Lock, Push-Type, Neo-Quick® or Other Mating Connector on the Inflatable Bladder

99% polypropylene beads mixed with 1% additive comprised of 51 wt. % poly-L-lactic acid (PLLA) polymer and 49 wt. % poly(butylene adipate-co-terephthalate) (PBAT), or a copolymer of the two for 5 minutes. The mixture/blend was then formed into polypropylene cuff connector and a nipple, luer lock, push-type, Neo-quick® or other mating connector on the inflatable bladder.

Polypropylene cuff connector and a nipple, luer lock, push-type, Neo-quick® or other mating connector on the inflatable bladder are formed through several steps which are described herein below:
Material Preparation: Polypropylene pellets or granules are selected as the raw material. These pellets are fed into the injection molding machine's hopper along with the additive.
Heating and Melting: The polypropylene pellets are heated and melted within the injection molding machine's barrel. The temperature is carefully controlled to ensure proper melting without causing degradation of the plastic material.
Injection: Once the polypropylene is molten, it is injected under high pressure into the mold cavity using a reciprocating screw or a plunger. The mold is closed and clamped to prevent any leakage during the injection process.
Cooling and Solidification: The molten polypropylene quickly starts to cool down as it comes into contact with the mold surfaces. Cooling channels within the mold help expedite the cooling process. As the plastic cools, it solidifies and takes on the shape of the buckle.
Mold Opening and Ejection: Once the polypropylene has solidified, the mold is opened, revealing the molded buckle. Ejection pins or mechanisms help push the cuff connector and a nipple, luer lock, push-type, Neo-quick® or other mating connector on the inflatable bladder out of the mold.
Trimming and Quality Control: Any excess material or flash around the edges of the buckle is trimmed off. The buckle is then inspected for defects, proper dimensions, and overall quality. This step ensures that the final product meets the required specifications.

### Example 3: Process of Making Nylon Straps

99 wt. % nylon was mixed/blended with 1 wt. % additive comprised of 51 wt. % poly-L-lactic acid (PLLA) polymer and 49 wt. % poly(butylene adipate-co-terephthalate) (PBAT), or a copolymer of the two for 5 minutes. The mixture/blend was then woven into nylon straps using the conventional method in the field.

The hardware produced by Example 2 and Example 3 were then assembled onto the main body fabric of Example 1 to provide a biodegradable hardware.

### Biodegradation Testing

The injection molded plastic was tested under standard ASTM D5511. This test method covers the determination of the degree and rate of anaerobic biodegradation of plastic materials in high-solids anaerobic conditions. The test materials are exposed to a methanogenic inoculum derived from anaerobic digesters operating only on pretreated household waste. The anaerobic decomposition takes place under high-solids (more than 30% total solids) and static non-mixed conditions. This test method is designed to yield a percentage of conversion of carbon in the sample to carbon in the gaseous form under conditions found in high-solids anaerobic digesters, treating municipal solid waste.

Anaerobic digested sewage sludge was mixed with household waste. To make the sludge adapted and stabilized during a short post-fermentation at 53° C., the sludge was pre-incubated (one week) at 53° C. This means that the concentrated inoculum was not fed but allowed to post ferment the remains of previously added organics allowing large easily biodegradable particles were degraded during this period and reduce the background level of biogas from the inoculums itself.

A sample of the anaerobic digested sewage sludge was analyzed for pH, percent dry solids, and volatile solids, as well as, the amount of CO2 and CH4 evolution during the testing. Table 1 lists the results of this initial testing.

Inoculum Medium: Remove enough inoculum (approximately 15 kg) from the post-fermentation vessel and mix carefully and consistently by hand in order to obtain a homogeneous medium. Test three replicates each of a blank (inoculum only), Positive control (Reference material) (thin-layer chromatography cellulose), negative control (optional), and the test substance being evaluated.

Manually mix 1000 g wet weight (at least 20% dry solids) of inoculum in a small container for a period of 2 to 3 min with 15 to 100 g of volatile solids of the test substance or the controls for each replicate. For the three blanks containing inoculum only, manually mix 1000 g of the same inoculum in a small container for a period of 2 to 3 min with the same intensity as was done for the other vessels containing test substance or controls. Determine the weight of the inoculum and test substance added to each individual Erlenmeyer flask accurately. Add the mixtures to a 2-L wide-mouth Erlenmeyer flask and gently spread and compact the material evenly in the flask to a uniform density.

After placing the Erlenmeyer flask in incubator, connect it with the gas collection device. Incubate the Erlenmeyer flasks in the dark or in diffused light at 52° C. (+2° C.) for thermophilic conditions, The incubation time shall be run until no net gas production is noted for at least five days from both the Positive control (Reference material) and test substance reactors. Control the pH of the water used to measure biogas production to less than two by adding HCl.

The most important biochemical characteristics of the inoculum such as pH, Volatile Fatty Acids, NH4+-N— and dry solids were studied.

TABLE 1
Results of Initial testing of the anaerobic digested sewage sludge

| Parameters | Requirement | Actual results |
|---|---|---|
| pH | 7.5 to 8.5 | 7.85 |
| Kjeldahl nitrogen | 0.5 to 2 g/kg wet weight | 1.46 |
| Dry Solids at 105° C. | >20% | 44.00 |
| Volatile Solids at 550° C. | Below 1 g/kg wet weight | 0.76 |

The biogas volume in the gas sampling bag was measured (Table-2). Presence of gas in the gas collector of Positive control indicated that the inoculum was viable and gas displacement was observed both in Positive control and Test Sample.

ASTM D5511 states that for the test to be considered valid, the Positive control must achieve 70% within 30 days with deviation less than 20% of the mean between the replicates.

Positive control (Reference material) showed 71.03% on 27th day with less than 20% of the mean difference between the replicates.

The gas displacement observed after 90 days is as shown in the table below.

TABLE 2
Biogas volume of the evolved gas during
the biodegradation process at 90 days

| Biodegradation | Total Volume |
|---|---|
| Test | 90 days (mL) |
| Inoculum | 2840 |
| Positive control (Reference material) | 10100 |
| Bioplastic | 6500 |

The percent biodegradation of Positive control (and Test sample was calculated by the measured cumulative carbon dioxide and methane production from each flask after subtracting carbon dioxide evolution and methane evolution from the blank samples at the end 90 days of testing. Calculations were based on Total Organic Carbon obtained of both Positive control and Test sample.

TABLE 3a
Percentage biodegradability of Test sample
with respect to Positive control

| | | Positive control | Biodegradable |
|---|---|---|---|
| | Inoculum | (Reference | plastic |
| Cellulose. Group | control | material) | Sample |
| Weight | 1000 ml | 10.2305 g | 10.1335 g |
| Total volume (ml) | 2840.00 | 10100.00 | 6500.00 |
| % CH4 | 13.20 | 44.90 | 21.80 |
| Volume of CH4 | 374.88 | 4534.90 | 1417.00 |
| (ml) | | | |
| weight of CH4 (g) | 0.2459 | 2.9749 | 0.9296 |
| % CO2 | 15.70 | 45.50 | 24.20 |
| Volume of CO2 | 445.88 | 4595.50 | 1573.00 |
| (ml) | | | |
| Weight of CO2 (g) | 0.8828 | 9.0991 | 3.1145 |
| Total weight of | 0.4228 | 4.6879 | 1.5381 |
| carbon in grams | | | |
| Theoretical weight | — | 4.3040 | 8.4999 |
| of carbon in grams | | | |
| (Ci) | | | |
| Biodegradation | — | 0.99097 | 0.13121 |
| % Biodegradation | — | 99.10 | 13.12 |

Based upon the above, the biodegradable polymer/plastic showed a 13.12% biodegration over 90 days. This satisfies the ASTM D5511 standards for biodegradation.

Although the present invention has been disclosed in the form of preferred embodiments and variations thereon, it will be understood that numerous additional modifications and variations could be made thereto without departing from the scope of the invention.

For the sake of clarity, it is to be understood that the use of “a” or “an” throughout this application does not exclude a plurality, and “comprising” does not exclude other steps or elements.

What is claimed is:

1. A blood pressure cuff comprising:
an inflatable bladder comprised of:
a non-biodegradable polymer selected from a group consisting of polyamide, polyester, polyethylene (PE), polypropylene (PP), polyvinylchloride (PVC), polyurethane and combinations thereof; and
an additive comprising a blend or copolymer of (1) a first polymer selected from a poly-lactic acid (PLA), a polyhydroxyalkanoates (PHA) or a combination thereof and (2) a second polymer which is a poly-terephthalate.

2. A blood pressure cuff comprising:
an inflatable bladder; and
at least one connector, wherein the inflatable bladder and the at least one connector are made from a material comprised of:
i. a non-biodegradable polymer selected from a group consisting of polyamide, polyester, polyethylene (PE), polypropylene (PP), polyvinylchloride (PVC), polyurethane and combinations thereof; and
ii. a biodegradable additive.

3. The blood pressure cuff of claim 2, wherein the inflatable bladder has a first hardness on the durometer scale and the at least one connector has a second hardness on the durometer scale, different than the first hardness on the durometer scale.

4. The blood pressure cuff of claim 2, wherein the inflatable bladder and at least one connector are made of the same material.

5. The blood pressure cuff of claim 2, wherein the inflatable bladder has a hardness of Shore A 50 to Shore 70 hardness.

6. The blood pressure cuff of claim 2, wherein the inflatable bladder has a Shore D 60 to Shore D 70 hardness.

7. The blood pressure cuff of claim 2, wherein the blood pressure cuff is a single-use disposable blood pressure cuff.

8. The blood pressure cuff of claim 2, wherein the blood pressure cuff further includes a sleeve and is reusable.

9. A blood pressure cuff comprising:
a. an inflatable bladder made of a non-biodegradable polymer and an additive, wherein the non-biodegradable polymer is selected from polyamide, polyester, polyethylene (PE), polypropylene (PP), polyvinylchloride (PVC), polyurethane and combinations thereof;
b. at least one connector made of a non-biodegradable polymer and an additive, wherein the non-biodegradable polymer is selected from a group consisting of nylon, polyester, polyethylene (PE), polypropylene (PP), polyvinylchloride (PVC), polyurethane and combinations thereof;
c. tubing made of a non-biodegradable polymer and an additive, wherein the non-biodegradable polymer is selected from a group consisting of polyvinyl chloride, polyethylene, polypropylene, polyurethane, polyamide, polytetrafluoroethylene, polyvinylidene fluoride, ethylene vinyl acetate, polycarbonate or combinations thereof; and
d. wherein the additive is added to each of the materials in a-c comprising a blend or copolymer of (1) a first polymer selected from a poly-lactic acid (PLA), a polyhydroxyalkanoates (PHA) or a combination thereof and (2) a second polymer which is a poly-terephthalate.

10. The blood pressure cuff of claim 9, wherein the non-biodegradable polymer in a-c is present in a concentration from 90-99.9 wt. %, the additive is present in a concentration from 0.1-10 wt. % and the additive has 30-70 wt. % of the first polymer and 30-70% of the second polymer.

11. The blood pressure cuff of claim 9, further comprising:
an antimicrobial additive.

12. The blood pressure cuff of claim 11, wherein the antimicrobial is selected from isothiazolinone treatments, zinc pyrithione, thiabendazole, and silver.

13. The blood pressure cuff of claim 9, wherein the inflatable bladder and at least one connector have different durometer hardnesses.

14. The blood pressure cuff of claim 9, wherein the blood pressure cuff is a single-use disposable blood pressure cuff.

15. The blood pressure cuff of claim 9, wherein the blood pressure cuff further includes a sleeve and is reusable.
